SEQUENCE LISTING (21 sequences)

SEQ ID NO: 1 - DNA, 85 bp, Sus scrofa
tcaaatctagtacgtttttgtaagcacaatgatgatgccaaacgacaaagagttactgcgatccttgataagctgataacaatga

SEQ ID NO: 2 - DNA, 90 bp, ARTIFICIAL
  NF1 HDR oligo to create R1947X
tcaaatctagtacgtttttgtaagcacaatgatgatgccaaatgaagcttcaaagagttactgcgatccttgataagctgataacaatga

SEQ ID NO: 3 - DNA, 85 bp, ARTIFICIAL
  NF1 HDR oligo for WT Clamp
tcaaatctagtacgtttttgtaagcacaatgacgacgctaaacggcaaagagttactgcgatcctcgacaaactcattacaatga

SEQ ID NO: 4 - protein, 34 aa, ARTIFICIAL
  LEFT TALEN RVD
NGNNNGNININNHDNIHDNININGNNNINGNNNI

SEQ ID NO: 5 - protein, 32 aa, ARTIFICIAL
  RIGHT TALEN RVD
HDNININNNNNINGHDNNHDNINNNGNINIHD

SEQ ID NO: 6 - protein, 34 aa, ARTIFICIAL
  LEFT TALEN RVD
NININNHDNIHDNININGNNNINGNNNINGNNHD

SEQ ID NO: 7 - protein, 32 aa, ARTIFICIAL
  RIGHT TALEN RVD
NGNINGHDNININNNNNINGHDNNHDNINNNG

SEQ ID NO: 8 - protein, 32 aa, ARTIFICIAL
  LEFT TALEN RVD
NNNINGNNNINGNNHDHDNININIHDNNNIHD

SEQ ID NO: 9 - protein, 32 aa, ARTIFICIAL
  RIGHT TALEN RVD
NGNINGHDNINNHDNGNGNINGHDNININNNN

SEQ ID NO: 10 - protein, 34 aa, ARTIFICIAL
  LEFT TALEN RVD
NNNNHDNIHDHDHDNNNGNNNGHDHDNNHDNNHD

SEQ ID NO: 11 - protein, 30 aa, ARTIFICIAL
  RIGHT TALEN RVD
HDNINGNNNGNIHDNGHDNGNNNIHDNGNG

SEQ ID NO: 12 - DNA, 85 bp, SUS SCROFA; identical to SEQ ID NO: 1

SEQ ID NO: 13 - DNA, 90 bp, ARTIFICIAL; identical to SEQ ID NO: 2
  ssNF1 HDR Oligo Sequence

SEQ ID NO: 14 - DNA, 85 bp, Sus scrofa
agctcgccacccccgcctggcacccgtgtccgcgccatggccatctacaagaagtcagagtacatgaccgaggtggtgaggcgct

SEQ ID NO: 15 - DNA, 90 bp, ARTIFICIAL
  ssTP53 HDR Oligo Sequence
agctcgccacccccgcctggcacccgggtccgcgccatggccatctaagcttaaagaagtcagagtacatgcccgaggtggtgaggcgct

SEQ ID NO: 16 - DNA, 23 bp, ARTIFICIAL
  PCR PRIMER
cctgcccccaccatcttcttatt

SEQ ID NO: 17 - DNA, 24 bp, ARTIFICIAL
  PCR PRIMER
gctctcgtacagtgctttgcacaa

SEQ ID NO: 18 - DNA, 24 bp, ARTIFICIAL
  PCR PRIMER
ctcccctgccctcaataagctgtt

SEQ ID NO: 19 - DNA, 21 bp, ARTIFICIAL
  PCR PRIMER
tgggaatgaggggtttggcag

SEQ ID NO: 20 - DNA, 20 bp, ARTIFICIAL
  PCR PRIMER-FORWARD
gtgggtcagctcgccacccc

SEQ ID NO: 21 - DNA, 20 bp, ARTIFICIAL
  PCR PRIMER - REVERSE
gtgggtcagctcgccacccc

(19) United States
(12) Patent Application Publication
(10) Pub. No.: US 2016/0160238 A1
(43) Pub. Date: Jun. 9, 2016

(54) HETEROZYGOUS MODIFICATIONS OF TUMOR SUPPRESSOR GENES

(71) Applicant: Recombinetics, Inc., Saint Paul, MN (US)

(72) Inventors: Adrienne Leigh Biggar, Eden Prairie, MN (US); Daniel F. Carlson, Woodbury, MN (US); Scott C. Fahrenkrug, Minneapolis, MN (US)

(21) Appl. No.: 14/938,295

(22) Filed: Nov. 11, 2015

Related U.S. Application Data
(60) Provisional application No. 62/078,857, filed on Nov. 12, 2014.

(51) Int. Cl. C12N 15/90; C12N 15/85

(52) U.S. Cl. CPC C12N 15/907; C12N 15/8509; C12N 2015/8527

(57) ABSTRACT

Animals genomically modified to have heterozygous modifications of one or more tumor suppressor genes are disclosed.

### CROSS REFERENCE TO RELATED APPLICATIONS

This application claims priority to U.S. Provisional Application No. 62/078,857 filed Nov. 12, 2014, which is hereby incorporated by reference in its entirety.

### TECHNICAL FIELD

The technical field relates to genetically modified animals.

### BACKGROUND

Neurofibromatosis Type 1 (NF1) is one of the most prevalent genetic disorders, occurring in one in every 3,000 live births, with over 100,000 affected people in the United States alone. NF1 is caused by somatic mutations in the Neurofibromin 1 (NF1) gene, in which 50% of cases are inherited, and 50% of cases are new mutations. NF1 is a debilitating disease, as patients often show skeletal abnormalities, scoliosis, learning disabilities, hypertension and epilepsy [1]. NF1 patients also have the potential to develop benign tumors known as neurofibromas throughout the peripheral nerves of their body. Although neurofibromas are benign, they can cause significant pain and mobility problems. Further, secondary genetic changes cause malignant transformation of neurofibromas in 10% of patients leading to the development of malignant peripheral nerve sheath tumors (MPNSTs) [2]. MPNSTs are highly aggressive and deadly sarcomas, and currently, the only treatment options for MPNSTs are either complete surgical resection or high-dose, non-specific chemotherapy [2]. Because of the close association with nerves, surgical resection is often not feasible, and chemotherapy often fails. While there has been considerable effort put forth in developing targeted therapies for these tumors, none have shown profound efficacy in the clinic and MPNSTs remain the leading cause of death for NF1 patients. NF1 patients also have a higher risk for the development of optic pathway gliomas, astrocytomas and juvenile myelomonocytic leukemia, in addition to multiple other tumor types [3].

### BRIEF DESCRIPTION OF THE DRAWINGS

FIG. 1: NF1 gene and protein alignment for human and swine. NF1 is highly conserved between human and swine. The swine exon 42 corresponds to the human exon 40, and the amino acid sequence in this region is also highly conserved. The amino acid arginine 1947 (R1947) is often mutated in human patients, and this amino acid is conserved in swine.

FIG. 2: TALEN deign to create the R1947X mutation in the NF1 pig gene. The pig NF1 exon 42 is at the top. Exon 42 is enlarged to show the TALEN binding sites of 3 TALEN pairs, and the Arginine 1947 (R1947) amino acid.

FIGS. 3A-3D: Utilizing TALENs and homology-dependent repair (HDR) to induce an R1947X mutation in the swine NF1 gene. 3A. The wild type (WT) sequence for exon 42 of the NF1 gene is shown at the top. A TALEN was designed for this locus (ssNF1 42.3 TALEN) and the TALEN binding sites are underlined (red). A 90 base pair HDR oligo was designed to induce the R1947X mutation (capitalized letters are changed base pairs from the WT sequence) and a novel restriction enzyme site (red, underlined) for RFLP analysis. The resulting R1947X allele is showed below. 3B. RFLP analysis of cell populations after 3 day incubation at 30 degrees Celsius shows that all three TALENs are able to induce HDR at varying rates, with ssNF1 42.1 being the most active (54.2% allele modification), ssNF1 42.2 being moderately active (45.1% allele modification) and ssNF1 42.3 being the least active of the 3 TALENs (27.2% allele modification). 3C. RFLP analysis for 24 clones shows the uncut (WT) band at 365 base pairs and the bands corresponding to the digested HDR allele at 191 and 174 base pairs. * Specifies heterozygous clones. 3D. Sequencing analysis for two clones demonstrate both a WT allele and an HDR allele.

FIGS. 4A-4C: TALEN activity correlates with the ability to isolate clones that are heterozygous for tumor suppressor genes. 4A. Graph shows percentage of clones that are WT, heterozygous, or homozygous by RFLP that were isolated from ssNF1 42.2 treated cells and compared to the predicted ratios. To predict the percentage of homozygous clones: (Day 3 RFLP activity)̂2. To predict the percentage of heterozygous clones: (Day 3 RFLP activity×2)(1-Day 3 RFLP activity). With ssNF1 42.2 which has higher activity than ssNF1 42.3, much more gene modification is observed, with a bias towards homozygous KO clones. 4B. With ssNF1 42.3, a TALEN pair with less activity, fewer homozygous KO clones are recovered, as expected, and the ratio of WT, heterozygous and homozygous clones were much closer to what would be predicted. 4C. Graph shows varying activity for ssNF1 42.2 and ssNF1 42.3 TALENs. 10 heterozygous clones by RFLP were chosen for sequencing analysis. ssNF1 42.2 which has much higher TALEN activity than ssNF1 42.3 resulted in 90% of the sequenced clones having indels in the WT allele, while only 30% of the clones from the ssNF1 42.3 TALEN treated cells showed indels.

FIG. 5: TALEN design for modifying NF1 and TP53 in cis in swine. NF1 and TP53 are located in close proximity on chromosome 12 in swine (chromosome 17 in human). TALENs were designed to create the R1947X mutation in NF1 and a Y155X mutation in TP53. TALEN binding sites relative to the desired mutations are shown above.

FIGS. 6A-6B: Utilizing TALENs and homology-dependent repair (HDR) to induce an R1947X mutation in the swine NF1 gene and an Y155X mutation in the swine TP53 gene. 6A. When co-transfected into primary swine fibroblasts, ssNF1 42.3 TALENs result in 25.5% allele modification by RFLP analysis and ssTP53 E6 TALENs result in 11.8% cutting by Cell analysis. 6B. RFLP analysis for 24 clones shows the uncut (WT) band and the bands corresponding to the digested HDR allele for both NF1 and TP53. * Specifies heterozygous clones. * Specifies clones that are heterozygous for both NF1 and TP53. RFLP analysis demonstrated that 8.9% of clones were heterozygous for NF1 by RFLP and 3.7% of clones were heterozygous for both NF1 and TP53 by RFLP, but all clones that were sequenced contained indels on the WT allele of either NF1 or TP53 or both.

FIG. 7: Melt curve for high definition melt analysis identifies clones heterozygous for TP53.

FIGS. 8A-8C: NF1 heterozygous pigs demonstrate increased Ras activity. 8A. Table showing outcome of SCNT experiments for Landrace Farm Pigs and Ossabaw Minipigs. 8B. Western blot of WT fibroblasts (left) and NF1 R1947X/+ fibroblasts (right). Staining shows increased activity of Ras in mutant cells. Cell lysates collected at 0, 5 and 15 minutes. 8C. Ras-GTP normalized to total Ras (top) or GAPDH (bottom). In both analyses, NF1 R1947X/+(NF1 Het) cells show increased Ras activity compared to NF1 WT cells.

FIGS. 9A-9D: NF1 heterozygous pigs have NF1-related phenotype. All of the NF1 R1947X/+Ossabaw minipigs born show hypopigmentation of varying degree. All of the piglets showed tan/brown hypopigmentation on their face (9A, top) and one piglet showed white hypopigmentation on its back (9A, bottom). 9B. Two of the NF1 R1947X/+Ossabaw minipigs showed signs of spine curvature and potential scoliosis upon necropsy both by gross observation (9B, left) and X-Ray imaging (9B, right). Scoliosis has been documented in about 20% of NF1 patients. 9C. Table identifying presence of multiple café au lait spots observed in one of many of the NF1 R1947X/+Ossabaw minipigs. 9D, 1-4 are photographs of the spots identified in 9C.

### DETAILED DESCRIPTION

While several mouse models have been developed to study NF1, the mouse model has limitations in terms of its power to model the human disease and is a poor model for the development of novel imaging techniques and surgical interventions. Herein, materials and methods are provided to establish a swine model of NF1 that can be used to better understand NF1 etiology, disease development and progression, the application of novel imaging and surgical techniques, and preclinical drug testing. Moreover, the model benefits from knocking out the TP53 gene, and embodiments of the same are presented.

In the course of this research, the inventors were, at first, unable to make a cell that was heterozygous for an NF1 and/or TP533 knockout. During the course of this research, the following theory of action was developed; the invention, however, is not to be limited to this theorized mechanism. It was appreciated that the problem is that NF1 and TP53 are tumor suppressor genes. Methods of making genetic modifications are studied primarily in cells and involve modifying cells and allowing them to replicate before being tested. The cells are livestock cells that are being used for making animals, by cloning. In this context, it is important to create methods that are effective with primary cells and with minimal replication and/or passaging of cells, which is a further challenge to making effective gene modifications. Since tumor suppressor genes are being knocked out, it was the cells that were homozygous for a knockout of a tumor suppressor gene were favored over cells that were heterozygous or wild-type.

With this realization, a few different approaches to making the modifications were attempted. There were additional constraints because one of the goals was to make livestock cells heterozygous, for knockout of both NF1 and TP53 in cis. One of these approaches was to cut the target tumor suppressor gene with targeted endonucleases and to provide two homology dependent repair (HDR) templates for the same gene. One of the HDR templates had the change that was intended to create the knockout. But the second HDR template had the wild type gene. This approach was effective, as per the Examples below.

Another approach also used two templates, with one of the templates being very close to having sequence identity with the wild type gene. But small changes were made to provide for easy detection of the presence of the almost-wild type HDR template. Silent mutations were made to provide for a novel restriction enzyme site. And the knocked-out allele was modified to have its own unique restriction site. Cells could then be tested to determine if both changes were present.

### Tumor Suppressor Genes

The working examples describe the modification of two different tumor suppressor genes. Accordingly, tumor suppressor genes may generally be modified, for instance:

### TP53 Tumor Protein p53

### PTEN Phosphatase and Tensin Homolog

### RBI (pRB or RB 1) Retinoblastoma 1

### Smad4 Smad Family Member 4

### BUBIB (BUB1) Budding Uninhibited by Benzimidazoles

BRCA1 Breast Cancer 1, early onset
BRCA2 Breast Cancer 2, early onset

### ST14 Suppressor of Tumorigenicity 14

pVHL Von Hippel-Lindau Tumor Suppressor

### CD95 Fas Receptor

### ST5 Suppressor of Tumorigenicity 5

### YPEL3 Yippee-like 3

### ST7 Suppressor of Tumorigenicity 7

### NF2 Neurofibromin 2 (Merlin)

### TSC1 Tuberous Sclerosis 1

### TSC2 Tuberous Sclerosis 2

### CDKN2A Cyclin-dependent Kinase Inhibitor 2A

### PTCH Patched

As described elsewhere, modifications may be disruption, knockout and suppression, with insertions/deletions (indels), frameshift, natural or wild type alleles, and so forth.

### Genetically Modified Animals

Animals may be made that are mono-allelic or bi-allelic for a chromosomal modification. For instance, the inventors have used methods of homologous dependent recombination (HDR) to make changes to, or insertion of exogenous genes into, chromosomes of animals. Tools such as TALENs and recombinase fusion proteins, as well as conventional methods, are discussed elsewhere herein. The inventors' laboratory has previously demonstrated exceptional cloning efficiency when cloning from polygenic populations of modified cells, and advocated for this approach to avoid variation in cloning efficiency by somatic cell nuclear transfer (SCNT) for isolated colonies (Carlson et al., 2011). Some have reduced this burden with sequential cycles of genetic modification and SCNT (Kuroiwa et al., 2004) however, this is both technically challenging and cost prohibitive. The ability to routinely generate bi-allelic KO cells prior to SCNT is a significant advancement in large animal genetic engineering. Bi-allelic knockout has been achieved in immortal cells lines using other processes such as ZFN and dilution cloning (Liu et al., 2010). Another group recently demonstrated bi-allelic KO of porcine GGTA1 using commercial ZFN reagents (Hauschild et al., 2011) where bi-allelic null cells could be enriched by FACS for the absence of a GGTA1-dependent surface epitope. While these studies demonstrate certain useful concepts, they do not show that animals or livestock could be modified because simple clonal dilution is generally not feasible for primary fibroblast isolates (fibroblasts grow poorly at low density) and biological enrichment for null cells is not available for the majority of genes.

Experimental results indicated that targeted nuclease systems were effectively cutting dsDNA at the intended cognate sites. Targeted nuclease systems include a motif that binds to the cognate DNA, either by protein-to-DNA binding, or by nucleic acid-to-DNA binding. The efficiencies reported herein are significant. The inventors have disclosed further techniques elsewhere that further increase these efficiencies.

Embodiments of the invention include a method of making a genetically modified animal, said method comprising exposing embryos or cells to a vector or an mRNA encoding a targeting nuclease (e.g., meganuclease, zinc finger, TALENs, guided RNAs, recombinase fusion molecules), with the targeting nuclease specifically binding to a target chromosomal site in the embryos or cells to create a change to a cellular chromosome, cloning the cells in a surrogate mother or implanting the embryos in a surrogate mother, with the surrogate mother thereby gestating an animal that is genetically modified without a reporter gene and only at the targeted chromosomal site. The targeted site may be one as set forth herein, e.g., the various genes described herein. Template-driven introgression methods are detailed herein. Embodiments of the invention include template-driven introgression, e.g., by HDR templates, to modify a tumor suppressor gene of a non-human animal, or a cell of any species.

This method, and methods generally herein, refer to cells and animals. These may be chosen from the group consisting non-human vertebrates, non-human primates, cattle, horse, swine, sheep, chicken, avian, rabbit, goats, dog, cat, laboratory animal, and fish. The term livestock means domesticated animals that are raised as commodities for food or biological material. The term artiodactyl means a hoofed mammal of the order Artiodactyla, which includes cattle, deer, camels, hippopotamuses, sheep, and goats that have an even number of toes, usually two or sometimes four, on each foot.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Ga14 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site. The examples herein have been performed with TALENs. Other embodiments are directed to the same processes and animals using other targeted endonucleases.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Application WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvC1, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-Mav L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255, U.S. 2012/0192298, U.S. 2011/0023159, and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001 and Kiwaki et al. Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. 89:6861, 1992, for a review of Cre/lox technology, and Brand and Dymecki, Dev. Cell 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. Nucleic Acids Res. 31:6873, 2003); Tol2 (Kawakami Genome Biology 8(Supp1.1):57, 2007; Minos (Pavlopoulos et al. Genome Biology 8(Supp1.1):52, 2007); Hsmar1 (Miskey et al.) Mol Cell Biol. 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA 82:6148-1652, 1985), gene targeting into embryonic stem cells (Thompson et al., Cell 56:313-321, 1989), electroporation of embryos (Lo, Mol. Cell. Biol. 3:1803-1814, 1983), sperm-mediated gene transfer (Lavitrano et al., Proc. Natl. Acad. Sci. USA 99:14230-14235, 2002; Lavitrano et al. Reprod. Fert. Develop. 18:19-23, 2006), and in vitro transformation of somatic cells, such as cumulus or mammary cells, or adult, fetal, or embryonic stem cells, followed by nuclear transplantation (Wilmut et al., Nature 385:810-813, 1997; and Wakayama et al., Nature 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO2. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×105 sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO2 atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science 280:1256-1258, 1998 and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; NY. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis Genetic Engineering News 12:1, 1992; Guatelli et al., Proc. Natl. Acad. Sci. USA 87:1874, 1990; and Weiss Science 254:1292, 1991. At the blastocyst stage, embryos can be individually processed for analysis by PCR, Southern hybridization and splinkerette PCR (see, e.g., Dupuy et al. Proc. Natl. Acad. Sci. USA 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature 391:806, 1998; Romano and Masino Mol. Microbiol. 6:3343, 1992; Cogoni et al., EMBO J. 15:3153, 1996; Cogoni and Masino Nature 399:166, 1999; Misquitta and Paterson Proc. Natl. Acad. Sci. USA 96:1451, 1999; and Kennerdell and Carthew Cell 95:1017, 1998. Constructs for shRNA can be produced as described by McIntyre and Fanning BMC Biotechnology 6:1, 2006. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

Embodiments include animals modified to express an RNAi that inhibits one or more tumor suppressor genes.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

Embodiments include animals modified to inducibly control one or more tumor suppressor genes. The control may be positive or negative, to turn on or to turn off.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function. Dominant negatives may be made to block a tumor suppressor gene.

### Founder Animals, Animal Lines, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. Pub. 2011/0059160 (U.S. Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for alteration of an indicated tumor suppressor allele. Such items can be used, for example, as research tools, or therapeutically.

### EXAMPLES

### Example 1

### Identification of Conserved Regions Between Pig and Human NF1 Genes to Select Target Sites

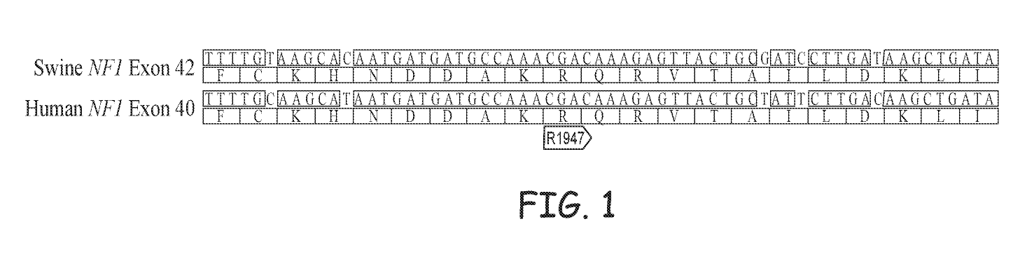

NF1 patients display a wide variety of mutations throughout the tumor suppressor gene Neurofibromin (NF1). A nonsense mutation (R1947X) has been identified as the most frequent alteration in NF1 patients, and accounts for one to two percent of all NF1 mutations [1]. R1947 is found in exon 42 of swine, which is highly conserved with exon 40 in humans, therefore we chose to engineer the R1947X mutation commonly found in humans into the swine genome to create a large animal model of NFL FIG. 1 is an alignment of the NF1 gene and protein for human and swine.

### Example 2

### Designing TALENs to Engineer a Heterozygous R1947X Mutation in the Swine NF1 Gene

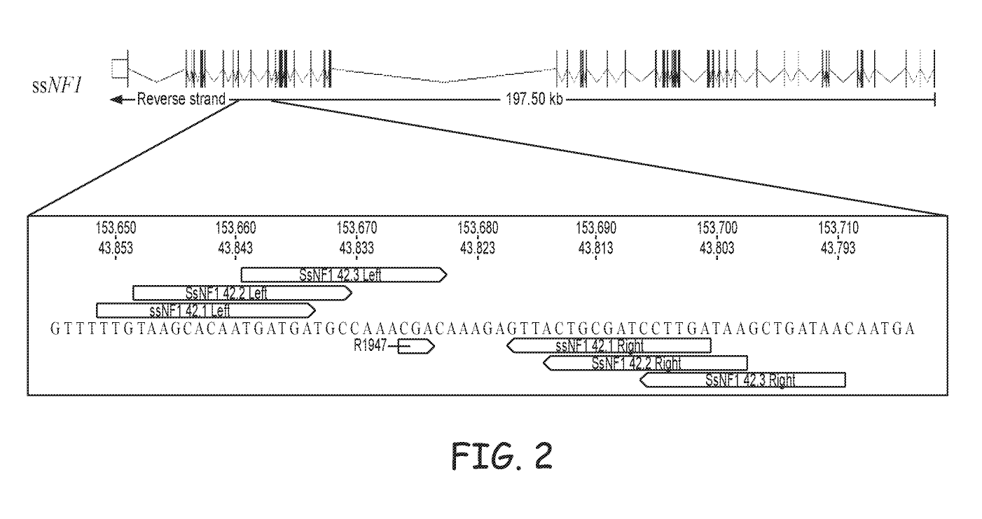

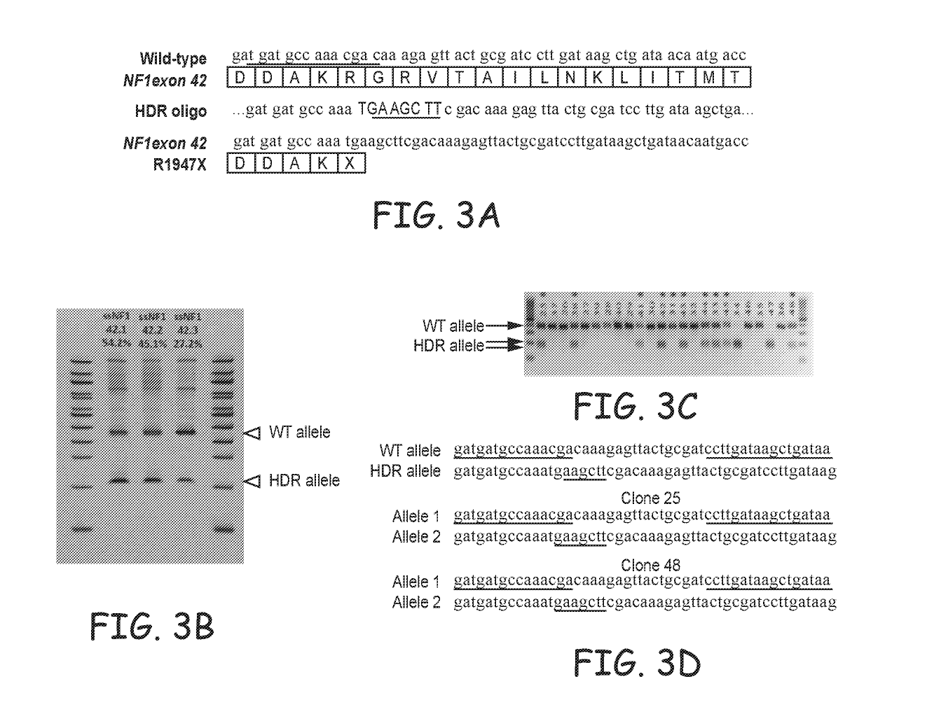

We chose to mimic the human NF1 R1947X mutation in pigs by TAL-effector nuclease (TALEN)-mediated homology-dependent repair (HDR), using a TALEN pair that targets exon 42 in the pig genome (corresponding to exon 40 in the human genome) of the NF1 gene, and an HDR construct to introduce a STOP codon, frame shift, and novel restriction enzyme site, allowing clones to be rapidly analyzed by restriction fragment length polymorphism (RFLP) analysis (FIGS. 2 and 3A). The three NF1 TALENs were transfected into Ossabaw pig primary fibroblasts along with the HDR oligo designed to induce the R1947X mutation, in addition to a novel RFLP site that changes the TALEN binding sites and prevents TALEN re-cutting (FIGS. 2 & 3A). After 3 days of incubation at 30 degrees Celsius, the cell populations were analyzed for the ability of each TALEN to induce HDR by RFLP analysis (FIG. 3B). This analysis showed that all three TALENs were active at varying rates (FIG. 3B). After treatment with the TALENs and HDR constructs, clones underwent (RFLP) analysis to identify modified alleles and to obtain heterozygous NF1 knockouts clones (FIG. 3C). Each of the resulting heterozygous clones by RFLP was sequenced to confirm the presence of both a wild-type and HDR allele, and can be used for chromatin transfer cloning techniques to generate NF^{R1947/+} swine (FIG. 3D).

### Example 3

### TALEN Activity Correlates with the Ability to Isolate Clones that are Heterozygous for Tumor Suppressor Genes

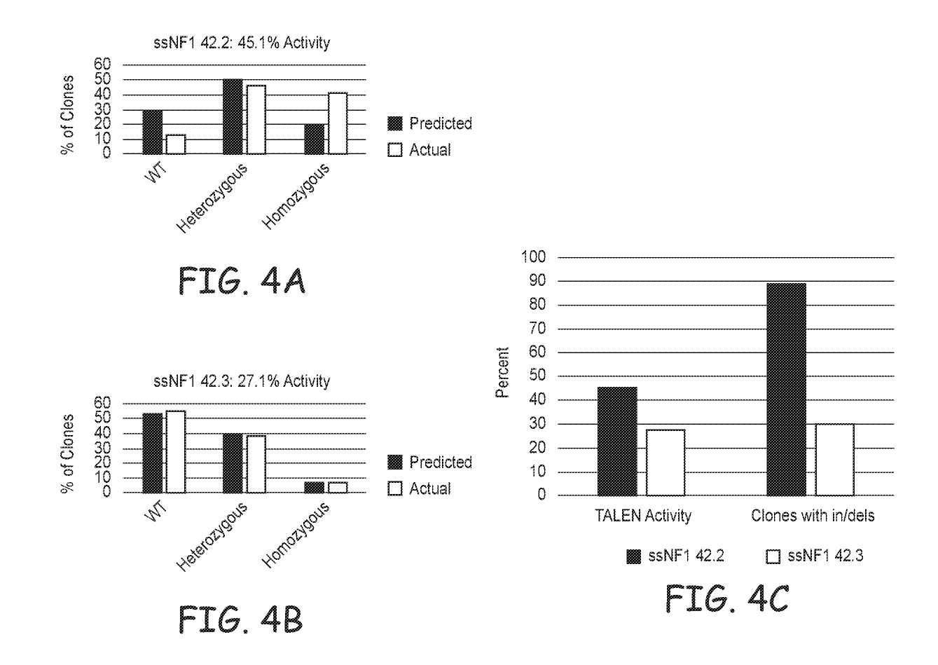

There is a selective advantage for loss of both copies of a tumor suppressor gene, so novel techniques are required for isolating clones heterozygous for NF1. The TALEN ssNF1 42.2 was chosen to use in an effort to create NF1 R1947X heterozygous clones due to its intermediate activity (45.1%) compared to ssNF1 42.1 (54.2%) and ssNF1 42.3 (27.2%) which we predicted may have activity that is too high or too low, respectively. When cells were treated with ssNF1 42.2, 41/89 (46.1%) of clones were shown to be heterozygous for NF1 R1947X by RFLP (FIG. 4A). Interestingly, 41.6% of the clones recovered were shown to be homozygous KOs, a much higher rate than would be predicted (20.3%, see figure legend for equations), suggesting there is a selective pressure to lose both NF1 alleles (FIG. 4A). This selective pressure to lose both copies of NF1 comes from the fact that NF1 is a tumor suppressor gene, so cells with homozygous loss likely have a growth and/or survival advantage over cells that are WT or heterozygous for NF1. Upon TOPO cloning and sequencing analysis, only 1/10 (10%) of the clones isolated from ssNF1 42.3 TALEN-treated cells were heterozygous for both the HDR allele and WT allele (FIG. 4C). 9/10 clones (90%) were heterozygous for the HDR allele and showed small insertions or deletions (indels) in the WT allele (FIG. 4C). We hypothesized that the indels seen in the WT allele may be due to both excess TALEN activity and the selective advantage for both copies of the gene to be non-functional, so we implemented ssNF1 42.3, a TALEN with only 27.2% activity as assessed by RFLP (FIG. 3B). ssNF1 42.3 resulted in a fewer number of clones that appeared heterozygous by RFLP as ssNF1 42.2 (35/91 clones, 38.5%), but the proportion of WT, heterozygotes, and homozygotes were quite similar to what would be predicted (FIG. 4B). When these clones were TOPO-cloned and each allele sequenced, it was found that only 3/10 clones (30%) were heterozygous for the HDR allele and showed small indels in the WT allele, while 7/10 (70%) of the clones were heterozygous for both the HDR allele and the WT allele (FIG. 4C). These experiments show that there is a selective advantage for homozygous knockout of tumor suppressor genes, and that TALENs with high activity will likely show modifications in both alleles by either HDR or indels. This technical challenge can be overcome by using TALENs with lower activity, as was demonstrated for NF1 (FIG. 4C).

### Example 4

### WT Clamp Method

Another method to address the technical challenge in creating cells that are heterozygous knockouts for a tumor suppressor gene is to concurrently use two HDR oligos to modify each allele of a gene separately, a method referred to as “WT Clamp”. One HDR oligo would induce the KO allele, and the other one would maintain the WT allele while preventing re-cutting and subsequent indels (Table 1). Based on the results from Example 1, it is clear that highly active TALENs have a tendency to cut both alleles of a gene. With the HDR oligo that was designed for NF1, re-cutting of the HDR-modified allele is prevented, due to changes in the TALEN binding sites when HDR occurs (FIG. 2A). It should be noted that re-cutting could also be prevented by inducing changes in the spacer lengths, although for NF1, this approach was not taken. In Example 1, ssNF1 42.2 is a highly active TALEN, which results in the recovery of heterozygous clones at a rate lower that would be expected (FIG. 4). Further, re-cutting of the WT allele results in a large proportion of clones containing indels (90%) (FIG. 4). While using TALENs with reduced activity, such as ssNF1 42.3, can help overcome this problem and result in a higher yield of heterozygous clones without indels on the WT allele (70%), a TALEN with reduced activity yields fewer clones that are heterozygous by RFLP to begin with, making the recovery of clones heterozygous for a tumor suppressor gene technically challenging (FIG. 4C). Further, there is no method to select for heterozygotes with the RFLP allele and an unmodified WT allele, therefore all clones must be TOPO-clones and sequenced, a time-consuming and resource-heavy approach.

TABLE 1
Designing HDR oligos
for WT Clamp method.

| | | RFLP site/ |
|---|---|---|
| Name | Sequence | SEQ ID NO. |
| NF1 WT sequence | tcaaatctagtacgtttttgta | SEQ ID 1 |
| | agcacaatgatgatgccaaacg | |
| | acaaagagttactgcgatcctt | |
| | gataagctgataacaatga | |
| NF1 HDR oligo to | tcaaatctagtacgtttttgta | HindIII |
| create R1947X | agcacaatgatgatgccaaatg | SEQ ID 2 |
| mutation | aA [symbol] [symbol] caaagagttactgc | |
| | gatccttgataagctgataaca | |
| | atga | |
| NF1 HDR oligo for | tcaaatctagtacgtttttgta | BceAI |
| WT Clamp (HDR- | agcacaatgacgacgctaaacg | SEQ ID 3 |
| WT allele) | gcaaagagttactgcgatcctc | |
| | gacaaactcattacaatga | |

Lower case bold- changed nucleotide;
capital letters- inserted nucleotides;
italicized sequence- RFLP site;
underlined sequence ssNF1 42.3 binding sites

To overcome this challenge we devised a new method by which re-cutting of the WT allele can be prevented by implementing a second HDR oligo that puts in silent nucleotide changes into the second allele, preventing re-cutting and subsequent indels, and creating a novel restriction enzyme site for more efficient screening of clones to identify heterozygotes. Table 1 provides examples demonstrating this method.

The WT clamp method takes advantage of the fact that TALENs are highly specific and modular, containing one RVD that binds a single nucleotide of the target sequence. By making silent mutation by changing the wobble base of each codon in the HDR, we can prevent TALENs from re-binding this locus after HDR has occurred, preventing indels in the HDR-WT allele. Further, by engineering specific silent base pair changes, the resulting HDR-WT allele will now contain a novel restriction enzyme site, in this case BceAI, which will allow us to screen through our colonies efficiently using RFLP. It should be noted that silent base pair changes were made using a swine codon usage database, to use codons that are used at similar levels for each amino acid. In applying this method, we would simply screen for colonies that have an allele that is cut with HindIII (representing the R1947X HDR allele) and an allele that is cut with BceA1 (representing the HDR-WT allele). This WT clamp method allows for more efficient isolation of colonies that are heterozygous for a tumor suppressor gene such as NFL

### Example 5

### Applying the WT Clamp Method to Multiple Genes

In cancer, it is often necessary to induce multiple mutations. In the case of NF1, two tumor suppressor genes often involved in disease progression, NF1 and TP53, are in close proximity (linked) on the same chromosome. Therefore, the inventors have designed experiments to knockout NF1 and TP53, two tumor suppressor genes, in cis, to predispose animals to malignancies seen in NF1 patients, including malignant peripheral nerve sheath tumors (MPNSTs). It is a common phenomenon for these genes to be heterozygously mutated in cis, and undergo loss of heterozygosity, resulting in cells that are null for both NF1 and TP53 [4-6]. This genetic change drives tumorogenesis of multiple types of malignancies and engineering this change in an animal model is critical to understanding this disease [4-6]. Multiplex gene editing in cis is very challenging due to the propensity to create large deletions between the target sites. We have previously demonstrated the occurrence of large deletions (6.5 kB) at the ssDMD locus occurring at a rate of 10.3% [7]. This example is further confounded by the fact that both targeted genes are tumor suppressor genes, thus requiring novel approaches to avoid homozygous loss of tumor suppressor genes and strategies to avoid large deletions by cutting the DNA in two locations near one another on a chromosome.

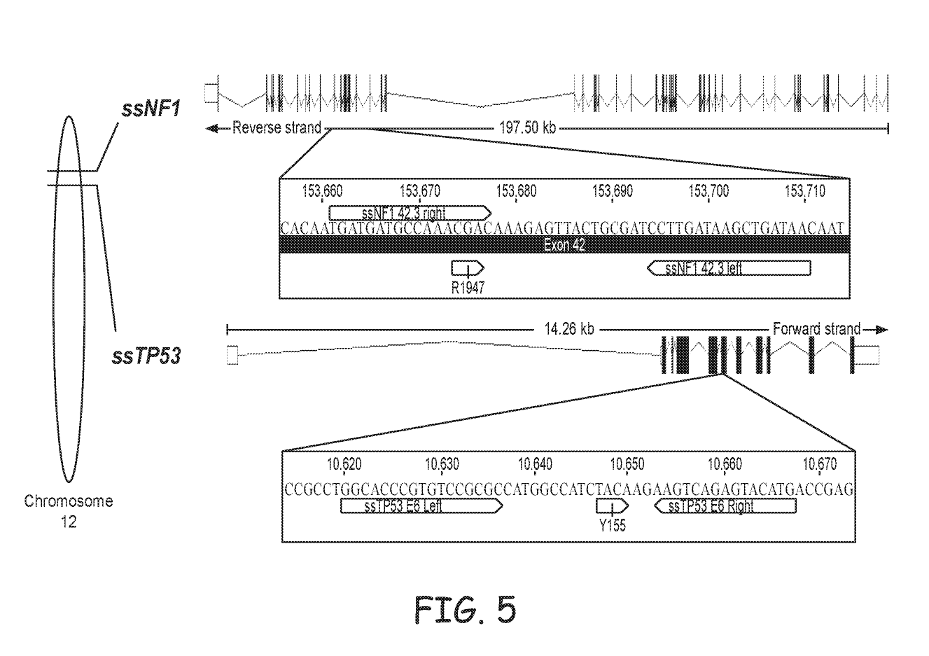

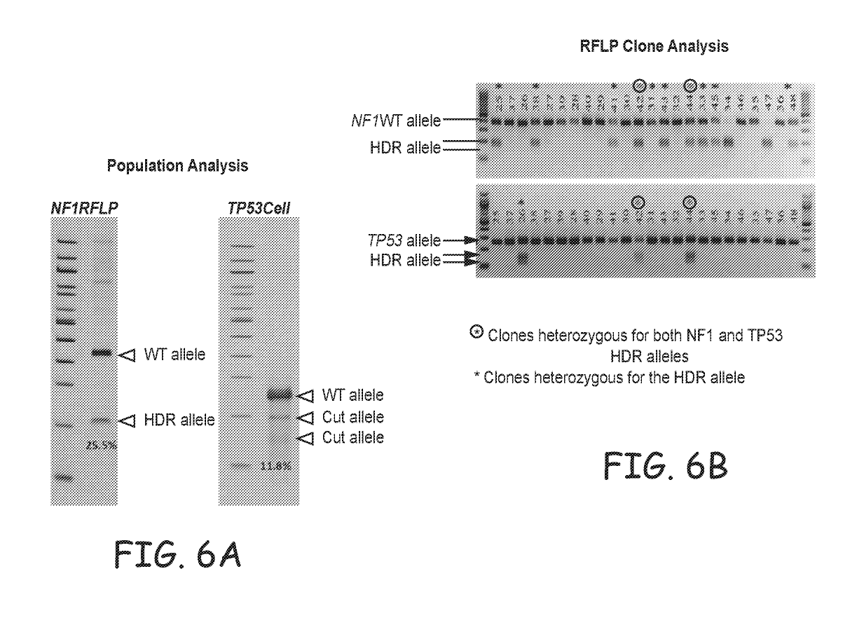

Similar methods as described above were used to develop and test TALENs and HDR oligos for the swine TP53 gene (ssTP53 E6), which is linked on chromosome 12 to the NF1 gene (FIG. 5) [7]. TALENs and HDR oligos that introduce nonsense mutations in the commonly mutated exon 6 of TP53 work at an efficiency of 17% [7]. When a multiplex approach was taken to create fibroblast lines with both NF1 and TP53 mutations, ssNF1 42.3 induced HDR at a rate of 25.5% and ssTP53 E6 cut at a rate of 11.8% (FIG. 6A). Using a multiplex approach where both ssNF1 42.3 and ssTP53 E6 were transfected into Ossabaw cells with their respective HDR oligos, we recovered NF1^{-/+}; TP53^{-/+} clones at a rate of 3.7% (7/190 clones) (FIG. 6B). Because ssTP53 E6 often fails to induce HDR, while still cutting at a rate high enough to induce indels, we performed a high definition melt analysis to determine if any of the TP53 alleles contained indels heterozygously in the absence of HDR. We identified 10 clones that were heterozygous for NF1 by RFLP and heterozygous by TP53 by high definition melt analysis for a total of 17 clones that were potentially heterozygous for both genes (see materials and methods). We sequenced all 17 clones that were heterozygous for both genes and found that 6/17 NF1 heterozygotes contained indels in the WT allele, 2/17 TP53 heterozygotes contained indels in the WT allele, and 7/10 TP53 heterozygotes that were identified by high definition melt analysis were in fact WT. In sum, 17/190 (8.9%) of clones were heterozygous for NF1 by RFLP; 7/190 (3.7%) clones were heterozygous for both NF1 and TP53 by RFLP; 17/17 clones contained indels on the wild type allele of either NF1 or TP53 or both. This resulted in no clones that were heterozygous for both NF1 and TP53.

To overcome the challenge of isolating clones that are heterozygous for both NF1 and TP53 we proposed the following approaches:

Reduce the activity of TALENs by using less TALEN mRNA in the transfection reaction or designing TALENs that have reduced activity.

1. Prevent recutting by reducing the time at 30 degrees Celsius.

2. Design HDR oligos for the wild-type allele that prevent re-cutting and subsequent indels, and allow efficient screening for the wild-type allele by RFLP (described in example 4).

Once clones are identified that are heterozygous for both NF1 and TP53, the identification of clones in which NF1 and TP53 mutations occur in cis will be done by radiation hybrid mapping, as previously described [8].

### Materials and Methods

### TALEN Design

Candidate TALEN target DNA sequences and RVD sequences were identified using the online tool “TAL Effector Nucleotide Targeter 2.0” as previously described [9]. TALEN target DNA sequences were chosen in sus scrofa exon 42 of the Neurofibromin (NF1) gene, which corresponds to homo sapiens exon 40 of the NF1 gene, where Arginine 1947 (R1947) is located. Input DNA sequences were 45 base pairs upstream and downstream of R1947. The R1947X nonsense mutation was chosen to mutate because it is the most frequent alteration observed in Neurofibromatosis Type 1 (NF1) patients [10]. TALENs designed to target the TP53 gene were designed in sus scrofa exon 6 to induce a Y155X mutation [11]. Table 2 provides a listing of the TALENs designed:

TABLE 2

| | Left | Right | | | |
|---|---|---|---|---|---|
| | TALEN | TALEN | | | |
| TALEN | Length/SEQ | Length/SEQ | Spacer | | |
| name | ID NO. | ID NO. | Length | Left TALEN RVDs | Right TALEN RVDs |
| ssNF1ex42.1 | 17 | 16 | 16 | NG NN NG NI NI | HD NI NI NN NN |
| | SEQ ID NO. 4 | SEQ ID NO. 5 | | NN HD NI HD NI | NI NG HD NN HD |
| | | | | NI NG NN NI NG | NI NN NG NI NI |
| | | | | NN NI | HD |
| ssNF1ex42.2 | 17 | 16 | 16 | NI NI NN HD NI | NG NI NG HD NI |
| | SEQ ID NO. 6 | SEQ ID NO. 7 | | HD NI NI NG NN | NI NN NN NI NG |
| | | | | NI NG NN NI NG | HD NN HD NI NN |
| | | | | NN HD | NG |
| ssNF1ex42.3 | 16 | 16/ | 16 | NN NI NG NN NI | NG NI NG HD NI |
| | SEQ ID NO. 8 | SEQ ID NO. 9 | | NG NN HD HD NI | NN HD NG NG NI |
| | | | | NI NI HD NN NI | NG HD NI NI NN |
| | | | | HD | NN |
| ssTP53 E6 | 17 | 15 | 16 | NN NN HD NI HD | HD NI NG NN NG |
| | SEQ ID NO. 10 | SEQ ID NO. 11 | | HD HD NN NG NN | NI HD NG HD NG |
| | | | | NG HD HD NN HD | NN NI HD NG NG |
| | | | | NN HD | |

### Donor Repair Template Design

A homology-dependent repair (HDR) oligo was designed to engineer an R1947X mutation in the NF1 gene. This HDR oligo contained 82 base pairs of homologous sequence to sus scrofa NF1 exon 42, a C→T mutation resulting in an R→X amino acid change, and a novel HindIII restriction enzyme site (AAGCTT), allowing for a facile restriction length polymorphism (RFLP) assay to be performed on clones to determine whether homologous recombination had in fact occurred. An HDR oligo was designed to engineer a Y155X mutation in the TP53 gene. This HDR oligo contains 83 base pairs of homologous sequence to sus scrofa TP53 exon 6, two base pair changes flanking the TALEN binding site, a C→T mutation resulting in a Y→X amino acid change, and a novel HindIII restriction enzyme site (AAGCTT), allowing for an RFLP assay to be performed on clones to determine whether homologous recombination had in fact occurred. These 90 mer oligonucleotide templates were synthesized by Integrated DNA Technologies, 100 nmole synthesis, purified by standard desalting, and resuspended to 400 uM Tris-EDTA. The HDR oligos that were designed are shown below in Table 3, with bold font denoting changes from the wild type (WT) sequence and the capitalized letters representing the introduced residues constituting the restriction site.

TABLE 3
HDR Oligos That Were
Designed For Repair Template

| | |
|---|---|
| ssNF1 Wild Type | tcaaatctagtacgtttttgtaagcacaatgatg |
| Sequence/ | atgccaaacgacaaagagttactgcgatccttga |
| SEQ ID NO. 12 | taagctgataacaatga |
| ssNF1 HDR Oligo | tcaaatctagtacgtttttgtaagcacaatgatg |
| Sequence/ | atgccaaatgaAGCTTcaaagagttactgcgatc |
| SEQ ID NO. 13 | cttgataagctgataacaatga |
| ssTP53 Wild Type | agctcgccacccccgcctggcacccgtgtccgcg |
| Sequence/ | ccatggccatctacaagaagtcagagtacatgac |
| SEQ ID NO. 14 | cgaggtggtgaggcgct |
| ssTP53 HDR Oligo | agctcgccacccccgcctggcacccgggtccgcg |
| Sequence/ | ccatggccatctaAGCTTAaagaagtcagagtac |
| SEQ ID NO. 15 | atgCccgaggtggtgaggcgct |

### TALEN Production

TALENs were produced as previously described [9]. Plasmids were constructed following the Golden Gate Assembly protocol using RCIscript-GoldyTALEN (Addgene ID 38143) as the final destination vector [12]. Assembled RCIscript vectors were prepared using QIAPREP SPIN MINIPREP kit (Qiagen), linearized by SacI, and used as template for in vitro TALEN mRNA transcription using mMESSAGE mMACHINE® T3 Kit (Ambion).

### Tissue Culture and Transfection

Pig fibroblasts were maintained at 37 or 30 degrees Celsius (as indicated) at 5% CO2 in DMEM supplemented with 10% fetal bovine serum, 100 I.U./mL penicillin and streptomycin, 2 mM L-Glutamine, 10 mM Hepes, 5 ug/mL apo-transferrin, 25 ng/uL rhEGF, and 20 ng/uL rhIGF. The Neon Transfection system (Life Technologies) was used to deliver TALENs and HDR oligos. Low passage Ossabaw or Landrace pig fibroblasts at 70-100% confluency were spilt 1:2 and harvested the next day at 70-80% confluency. Approximately 600,000 cells were resuspended in “R” Buffer (Life Technologies) with mRNA TALENs and HDR oligos and electroportated in 100 uL tips using the following parameters: input voltage: 1800V; pulse width: 20 ms; pulse number: 1. 0.5-2 ug of TALEN mRNA and 0.1-0.4 nmol of HDR oligos for the specific gene(s) of interest were included for each transfection. Transfected cells were cultured for 2 or 3 days at 30 degrees Celsius, and then analyzed for gene editing efficiency and plated for colonies.

### Clone Derivation

Two or three days post transfection, 50 to 250 cells were seeded onto 10 cm dishes and cultured until individual colonies reached circa 5 mm in diameter. 8 mL of a 1:4 (vol/vol) mixture of TrypLE and DMEM media (Life Technologies) was added and colonies were aspirated, transferred into wells of a 48-well dish and a replica 96 well dish and cultured under the same conditions. Colonies reaching confluence were collected for cryopreservation and sample preparation for genotyping.

### Cryopreservation

Samples were prepared for cryopreservation by spinning down cells and resuspending in cryopreservation media made of 90% fetal bovine serum (Atlas) and 10% dimethyl sulfoxide (Sigma). Samples were initially frozen down at −80 degrees Celsius for 4 hours and transferred to liquid nitrogen for long-term storage.

### Sample Preparation

Transfected cells populations at day 3 were collected from a well of a 6-well dish and approximately 10% were resuspended in 20 μl of 1×PCR compatible lysis buffer: 10 mM Tris-Cl pH 8.0, 2 mM EDTA, 0.45% Triton X-100 (vol/vol), 0.45% Tween-20 (vol/vol) freshly supplemented with 200 μg/ml Proteinase K. The lysates were processed in a thermal cycler using the following program: 55° C. for 60 minutes, 95° C. for 15 minutes. Colony samples from dilution cloning were treated as above using 20-30 μl of lysis buffer.

### Surveyor Mutation Detection and RFLP Analysis

PCR flanking the intended sites was conducted using Platinum Taq DNA polymerase HiFi (Life Technologies) with 1 μl of the cell lysate according to the manufacturer's recommendations. Primers for each site are listed in Table 4. The frequency of mutation in a population was analyzed with the SURVEYOR MUTATION DETECTION KIT (Transgenomic) according to the manufacturer's recommendations using 10 ul of the PCR product as described above. RFLP analysis was performed on 10 μl of the above PCR reaction using the restriction enzyme indicated in the Table 5. Surveyor and RFLP reactions were resolved on a 10% TBE polyacrylamide gels and visualized by ethidium bromide staining. Densitometry measurements of the bands were performed using ImageJ; and mutation rate of Surveyor reactions was calculated as previously described [13]. Percent HDR was calculated via dividing the sum intensity of RFLP fragments by the sum intensity of the parental band+RFLP fragments. RFLP analysis of colonies was treated similarly except that the PCR products were amplified by 1× ACCUSTART II GELTRACK SUPERMIX (Quanta Biosciences) and resolved on 2.5% agarose gels.

TABLE 4
Primers Used For Mutation Detection

| | | Forward | Reverse |
|---|---|---|---|
| Gene | Amplicon | Primer | Primer |
| ssNF1 | 365 base | CCTGCCCCCACCA | GCTCTCGTACAGT |
| | pairs | TCTTCTTATT/ | GCTTTGCACAA/ |
| | | SEQ ID NO. 16 | SEQ ID NO. 17 |
| ssTP53 | 251 base | CTCCCCTGCCCTC | TGGGAATGAGGGG |
| | pairs | AATAAGCTGTT/ | TTTGGCAG/ |
| | | SEQ ID NO. 18 | SEQ ID NO. 19 |

TABLE 5
Restriction Enzymes Used for HDR Detection

| | | Restriction enzyme site | |
|---|---|---|---|
| Gene | Amplicon | induced by HDR | Product size with HDR |
| ssNF1 | 365 bps | HindIII | ssNF1 42.2: 174 bps + |
| | | | 191 bps |
| | | | ssNF1 42.3: 182 bps + |
| | | | 183 bps |
| ssTP53 | 251 bps | HindIII | ssTP53 E6: 106 bps + |
| | | | 145 bps |

### High Definition Melt Analysis

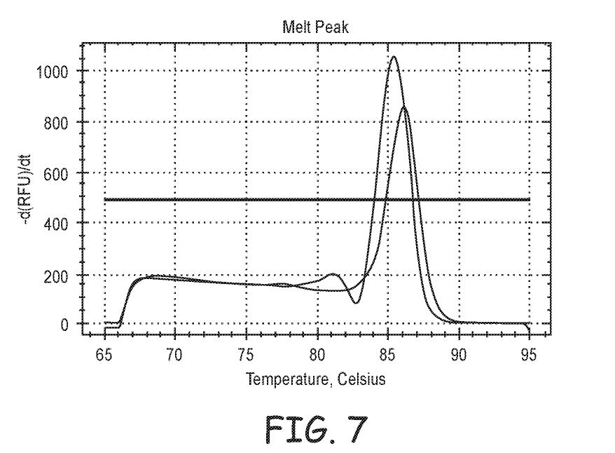

High definition melt analysis was done using 2 uL of cell lysis (as described above) diluted 1:10, 2 uM of high definition melt primers (Table 6), 1× PRECISION MELT SUPERMIX (BioRad). Reactions were run in a BioRad CFX Connect machine using a 2 step protocol with an annealing temperature of 55 degrees and 40 cycles. Melt curves were analyzed using BioRad's CFX Manager high definition melt analysis. Heterozygous clones were identified by the characteristic profile shown in FIG. 7.

TABLE 6
Primers Used for High
Definition Melt Analysis

| | | Forward | Reverse |
|---|---|---|---|
| Gene | Amplicon | Primer | Primer |
| ssTP53 | 96 bps | gtgggtcagctcg | ggacagcgcctca |
| | | ccacccc/ | ccacctc/ |
| | | SEQ ID NO. 20 | SEQ ID NO. 21 |

### Amplicon Sequencing and Analysis

DNA was isolated from transfected populations and cloned and amplified by 1× ACCUSTART II GELTRACK SUPERMIX (Quanta Biosciences). A portion of the PCR product was resolved on a 2.5% agarose gel to confirm size prior to PCR cleanup using the MinElute PCR Purification Kit (Qiagen). Samples were submitted to the University of Minnesota Genomics Center to be sequenced using standard Sanger sequencing. For samples in which the alleles were heterozygous, fresh PCR product was TOPO-TA cloned into pCR4 (Life Technologies), and individual TOPO clones were used as template for a second PCR reaction to amplify the region of interest prior to PCR cleanup using the MINELUTE PCR PURIFICATION KIT (Qiagen).

### Example 6

### Production of Nf1 Heterozygous Pigs Demonstrating Ras Hyperactivity

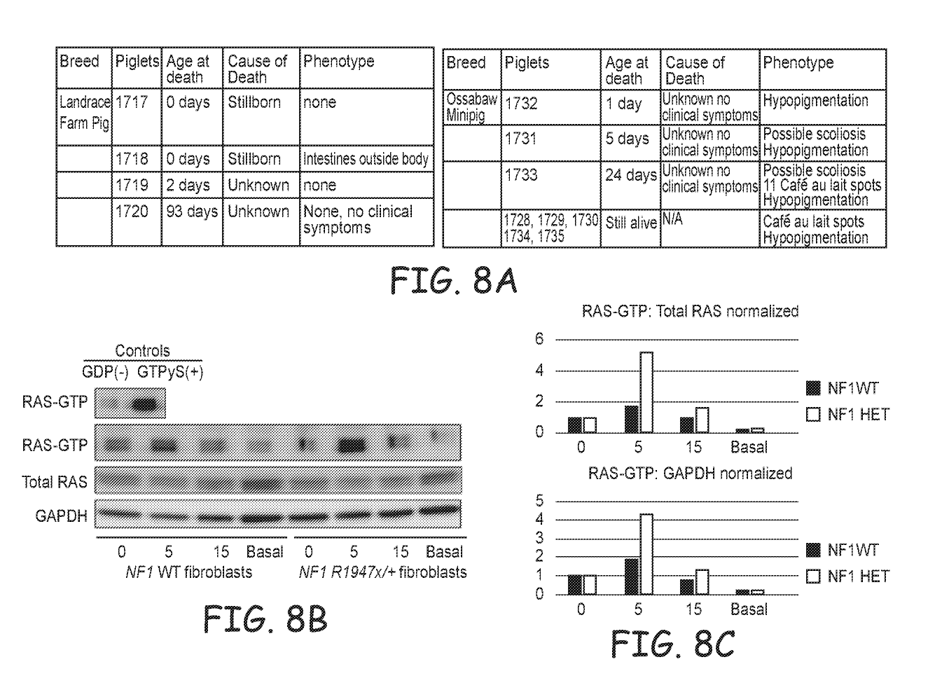

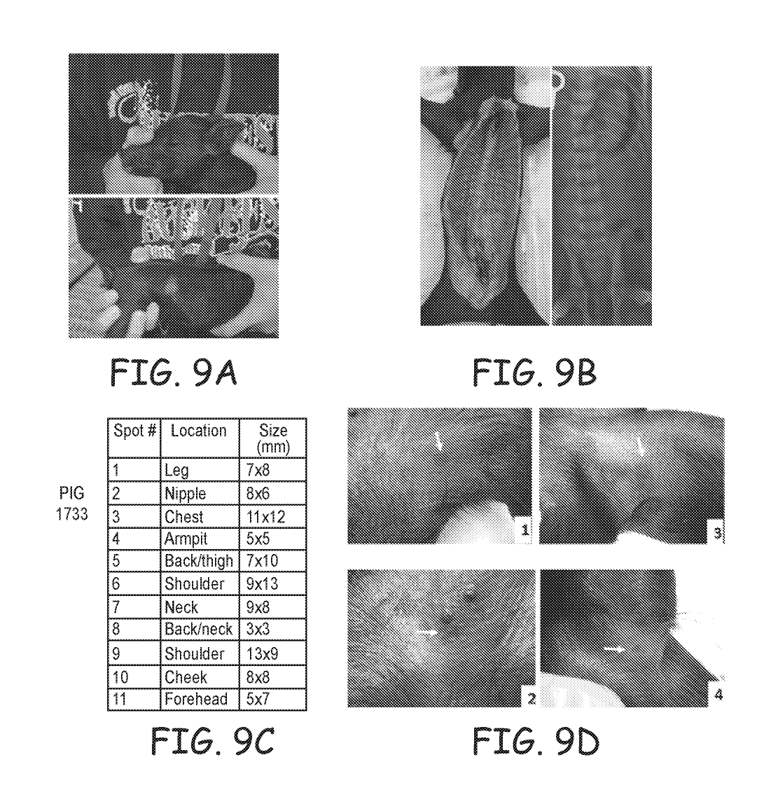

Three rounds of somatic cell nuclear transfer were performed on male Landrace cells, heterozygous for the R1947X mutation in the NF1 gene. From three rounds of cloning and embryo transfer, one pregnancy was established from which 4 piglets were born—2 were stillborn, one died at 2 days old, one that died at 93 days old, FIG. 8A. Surprisingly, there were no NF1-related phenotypes observed in the Landrace genetic background. Subsequently, three rounds of somatic cell nuclear transfer were performed male Ossabaw minipig cells heterozygous for the R1947X mutation in the NF1 gene. From three rounds of cloning and embryo transfer, two pregnancies were established and 8 total piglets were born (3 from one litter and 5 from another litter), FIG. 9A. Three piglets have died at 1, 5, and 24 days. Multiple animals have shown café au lait spots and signs of potential scoliosis, FIGS. 9A-9D. Five NF1 R1947X/+Ossabaw minipigs remain alive.

Active Ras (Ras-GTP) was quantified using an active Ras pulldown kit (ThermoFisher) in which GTP-bound Ras is pulled down with a GST-Raf1 Ras-binding domain tethered to a glutathione agarose resin. Cells were serum starved for 12 hours prior to serum stimulation and cell lysates were collected at 0, 5, and 15 minutes following serum stimulation, as well as at basal serum stimulation (not serum-starved). Both NF1 wild type (WT) and NF1 heterozygous (R1947X/+) cells show a peak in Ras-GTP levels 5 min following serum starvation, FIG. 8B. When Ras-GTP was normalized to either total Ras (FIG. 8C, top) or GAPDH (FIG. 8C, bottom), NF1 R1947X/+(NF1 Het) cells show increased Ras activity compared to NF1 WT cells. Ras-GTP levels remain higher for NF1 R1947X/+(NF1 Het) cells compared to NF1 WT cells 15 minutes after serum stimulation. These results show that the R1947X mutation identified in humans and transferred to swine is indeed involved in the etiotolgy of neurofibromatosis. Further, it shows that Ras plays an integral part in this pathology. Further, it shows that NF1 mutations play a role Ras hyperactivity.

NF1 heterozygous pigs have NF1-related phenotypes: FIG. 9A-9C provide examples of phenotypes of NF1 R1947X/+Ossabaw minipigs born showing phenotypes of neurofibromatosis. FIG. 9A, tan/brown hypopigmentation of face (top) and white hypopigmentation on back (bottom) of NF1 R1947X/+Ossabaw minipigs. FIG. 9B, Two of the NF1 R1947X/+Ossabaw minipigs showed signs of spine curvature and potential scoliosis upon necropsy both by gross observation (left) and X-Ray imaging (right). FIG. 9C, Café au lait spots have been observed in many of the NF1 R1947X/+Ossabaw minipigs. The table (left) describes 11 café au lait spots seen in a single animal, with photographs of the lesions as indicated. Café au lait spots are extremely common in NF1 patients and are used as criteria for diagnosing NF1 in humans. FIG. 9D, 1-4 are photographs of the spots corresponding to 1-4 of 9C.

### Example 7

### Identification of SNPs to Determine Whether Induced Mutations in NF1 and TP53 are in Cis or Trans

The inventors have identified potential SNPs within 5,100 base pairs of the NF1 R1947X or TP53 S119X mutations induced by TALEN-mediated homology-dependent repair (Tables 7 and 8). One or more of the identified SNPs will be used to identify which chromosome the NF1 R1947X mutation occurred on, and which chromosome the TP53 S119X mutation occurred on. With the data collected from a radiation hybrid map of this loci on chromosome 12 that harbors the NF1 and TP53 genes, the inventors will be able to identify clones in which the NF1 R1947X and TP53 S119X mutations occur on the same chromosome.

TABLE 7
NF1

| | Location from | | | | |
|---|---|---|---|---|---|
| SNP | NF1 R1974X | Genetic | % | Seen in | |
| Name | (bps) | Change | TOPO | population | notes |
| 16-314 | 5028 | A/G | 0.63 | YES | intron |
| 16-515 | 4827 | T/C | 0.63 | YES | intron |
| 12-4192 | 1150 | G/A | 0.75 | NO | intron |
| 11-4895 | 447 | A/G | 0.25 | NO | intron |
| 11-5397 | 59 | T/C | 0.25 | NO | in exon 42 |
| | | | | | missense |
| 17-6040 | 702 | G/T | 0.63 | NO | intron |

TABLE 8
TP53

| | Location from | | | | |
|---|---|---|---|---|---|
| SNP | TP53 S119 | Genetic | % | Seen in | |
| Name | (bps) | Change | TOPO | population | notes |
| 16-661 | 4722 | A/G | 0.5 | YES | intron |
| 16-762 | 4621 | T/C | 0.5 | NO | intron |
| 16-765 | 4618 | G/A | 0.5 | YES | intron |
| 16-805 | 4579 | C/T | 0.5 | YES | intron |
| 16-983 | 4400 | T/C | 0.5 | NO | intron |
| 15-1214 | 4167 | G/A | 0.75 | YES | intron |
| 15-1222 | 4159 | G/A | 0.75 | NO | intron |
| 15-1284 | 4097 | G/T | 0.625 | YES | intron |
| 15-1304 | 4077 | T/C | 0.75 | NO | intron |
| 15-1559 | 3822 | G/T | 0.375 | YES | intron |
| 15-1683 | 3698 | A/G | 0.375 | NO | intron |
| 15-1764 | 3617 | C/T | 0.375 | NO | intron |
| 14-2068 | 3313 | A/G | 0.625 | YES | intron |
| 14-2082 | 3299 | G/A | 0.625 | YES | intron |
| 14-2423 | 2958 | T/C | 0.5 | YES | intron |
| 13-3021 | 2360 | C/G | 0.375 | NO | intron |
| 13-3059 | 2322 | G/A | 0.375 | NO | intron |
| 13-3079 | 2302 | G/A | 0.375 | NO | intron |
| 13-3177 | 2204 | T/A | 0.375 | NO | intron |
| 11-4961 | 420 | A/G | 0.333333 | NO | intron |
| 11-5152 | 229 | T/C | 0.666667 | YES | intron |
| 11-5210 | 171 | T/G | 0.666667 | YES | intron |

The following paragraphs enumerated consecutively from 1 through 50 provide for various aspects of the present disclosure. In one embodiment, in a first paragraph (1), the present disclosure provides:

1. A swine or a cell or an embryo comprising a genomically modified NF1 gene and/or a modified TP53 gene.
2. The swine or cell or embryo of paragraph 1, wherein the modified NF1 gene comprises a modification at a location that is the equivalent of the arginine 1947 in human.
3. The swine or cell or embryo of paragraphs 1 and 2, wherein the modified NF1 gene and/or the modified TP53 gene is modified to include a premature stop codon.
4. The swine or cell or embryo of any of paragraphs 1-3, having a heterozygous modification of the NF1 gene.
5. The swine or cell or embryo of any of paragraphs 1-4, having a heterozygous modification of the TP53 gene.
6. The swine or cell or embryo of any of paragraphs 1-5, having a modification of both the NF1 gene and the TP53 gene.
7. The swine or cell or embryo of paragraphs 1-6, wherein the modifications are in cis.
8. The swine or cell or embryo of any of paragraphs 1-7, wherein one allele of the NF1 gene is a wildtype allele.
9. The swine or cell or embryo of any of paragraphs 1-8, wherein one allele of the TP53 gene is a wildtype allele.
10. The swine or cell or embryo of any of paragraphs 1-9, wherein one allele of the NF1 gene is a wildtype allele, except the wildtype NF1 allele has at least one silent mutation. Alternatively: has only 1, 2, 3, 4, or 5 silent mutations.
11. The swine or cell or embryo of any of paragraphs 1-10, wherein one allele of the TP53 gene is a wildtype allele, except the wildtype TP53 allele has at least one silent mutation. Alternatively: has only 1, 2, 3, 4, or 5 silent mutations.
12. The swine or cell or embryo of paragraphs 1-11, wherein the silent mutation provides a site of attack for a restriction enzyme. Example: 1, 2, 3, 4, 5 silent mutations that provide 1, 2, 3, 4, or 5 sites, with the sites being specific to a single enzyme or providing sites for a plurality of restriction enzymes.
13. The swine or cell or embryo of any of paragraphs 1-12 having a modification (silent or otherwise) that, because of the modification, provides a site of attack for a restriction enzyme. Example: 1, 2, 3, 4, 5 mutations that provide 1, 2, 3, 4, or 5 sites, with the sites being specific to a single enzyme or providing sites for a plurality of restriction enzymes.
14. The swine or cell or embryo of paragraphs 1-13, being a miniature pig and/or ossabaw pig and/or landrace pig and/or founder and/or F1.
15. The cell of paragraphs 1-14, being primary and/or swine and/or low passage (less than 13 passages).
16. The cell of paragraphs 1-15, being a zygote, oocyte, gamete, sperm, or a member of an embryo/blastomere.
17. A method of making any of paragraphs 1-16, comprising use of a targeted endonuclease and/or homology dependent repair template. The cell may be used to make the animal, e.g., by cloning.
18. A method of making an animal, cell, or embryo comprising introducing into a cell or an embryo:

a targeted endonuclease directed to a target chromosomal DNA site,

a first HDR template homologous to the target chromosomal DNA site that comprises a first sequence that is exogenous to the target chromosomal DNA site, and

a second HDR template homologous to the target chromosomal DNA site that comprises a second sequence that is exogenous to the target chromosomal DNA site.

19. The method of paragraph 18 wherein the second sequence is identical to the target DNA chromosomal site.
20. The method of paragraphs 18 and 19, wherein the second sequence has 99% identity to the target DNA chromosomal site. Alternatively, a value from 95 to 99.99%; artisans will immediately appreciate that all ranges and values within this range are contemplated and supported, e.g., 97, 99.8, at least 95%.
21. The method of paragraphs 18 through 20, wherein the second sequence is identical to the target DNA chromosomal site except for: (i) one or more silent mutations; (ii) a number of bases ranging from 1-5. For instance, the number of silent mutations may be from 1-5.
22. The method of any of paragraphs 18-21, wherein the second sequence is identical to the target DNA chromosomal site except for a change in sequence that allows for cleavage by a predetermined restriction enzyme. Further methods provide for any number of such changes, e.g., from 1-5.
23. The method of any of paragraphs 18-22, wherein the exogenous sequence comprises, or is: (i) an allele found in nature; (ii) an allele found in the same species; (iii) an allele found in another breed of the same species (an allele that is not in the same breed); (iv) an allele from a different species (an allele not from the same species); (v) a sequence that creates a knockout of a gene; (vi) an expressible selection marker (e.g., antibiotics, fluorescent protein); (viii) inducible promoter; (ix) landing pad; or (x) any combination of i-ix as may be appropriate.
24. The method of any of paragraphs 18-23 wherein the animal, cell, or animal is heterozygous for the genetic modification made by the first HDR template.
25. The method of any of paragraphs 18-24, applied to make a modified NF1 and/or TP53 site.
26. The method of any of paragraphs 18-24, applied to make a modified tumor suppressor gene.
27. The method of any of paragraphs 18-26, further comprising adjusting a ratio of the first HDR template to the second HDR template.
28. The method of any of paragraphs 18-27, with the first template comprising a first site for a first restriction enzyme and the second template comprising a second site for a second restriction enzyme. With said first site and second site being novel relative to the target site and/or target chromosome and/or breed and/or species and/or animal. Alternatively: addition sites for additional unique enzymes, so three or more restriction enzyme sites are present, for instance a number from 3 to 10; e.g., 4, 5, 6, 7, 8, 9.
29. The method of any of paragraphs 18-28, comprising screening a plurality of cells for a genetic modification comprising identifying cells that comprise the first site and the second site. Or from 3-10 sites.
30. An animal or a cell or an embryo comprising a genomically modified tumor suppressor gene, said gene being heterozygously modified.
31. The animal or cell or embryo of paragraph 30, made by a method of any of 18-30.
32. The animal or cell or embryo of any of paragraphs 30-31, wherein one allele of the modified gene is a wildtype allele, except the wildtype NF1 allele has at least one silent mutation. Alternatively: has only 1, 2, 3, 4, or 5 silent mutations.
33. The animal or cell or embryo of paragraphs 30-32, wherein the silent mutation provides a site of attack for a restriction enzyme. Example: 1, 2, 3, 4, 5 silent mutations that provide 1, 2, 3, 4, or 5 sites, with the sites being specific to a single enzyme or providing sites for a plurality of restriction enzymes.
34. The animal or cell or embryo of any of paragraphs 30-33, comprising a modification (silent or otherwise) at the gene (at one or both alleles of the gene after the modification is accomplished) that, because of the modification, provides a site of attack for a restriction enzyme. Example: 1, 2, 3, 4, 5 mutations that provide 1, 2, 3, 4, or 5 sites, with the sites being specific to a single enzyme or providing sites for a plurality of restriction enzymes.
35. The animal or cell or embryo of any of paragraphs 30-34, being a livestock, cattle, swine, a miniature pig and/or ossabaw pig and/or Landrace pig and/or founder and/or F1.
36. The cell of any of paragraphs 30-35, being primary and/or animal and/or low passage (less than 13 passages).
37. The cell of any of paragraphs 30-36, being a zygote, oocyte, gamete, sperm, or a member of an embryo/blastomere.
38. Use of a targeted endonuclease to genomically modify a cell, embryo or animal wherein the modification comprises a mutation to one or more tumor suppressor genes.
39. The use of any of the preceding paragraphs, wherein the modification further comprises

a first HDR template homologous to a target chromosomal DNA site that comprises a first sequence that is exogenous to the target chromosomal DNA site, and

a second HDR template homologous to the target chromosomal DNA site that comprises a second sequence that is exogenous to the target chromosomal DNA site.

40. The use of any of the preceding paragraphs, wherein the second sequence is identical to the target DNA chromosomal site.
41. The use of any of the preceding paragraphs, wherein the second sequence has 99% identity to the target DNA chromosomal site.
42. The use of any of the preceding paragraphs, wherein the second sequence has a 95% identity to the target DNA chromosomal site.
43. The use of any of the preceding paragraphs, wherein the second sequence is identical to the target DNA chromosomal site except for one or more silent mutations.
44. The use of any of the preceding paragraphs, wherein the number of bases changed in the one or more silent mutations is from 1-6.
45. The use of any of the preceding paragraphs, wherein the second sequence is identical to the target DNA chromosomal site except for a change in sequence that allows for cleavage by one or more restriction enzymes.
46. The use of any of the preceding paragraphs, wherein the exogenous sequence comprises: (i) an allele found in nature; (ii) an allele found in the same species; (iii) an allele found in another breed of the same species; (iv) an allele from a different species; (v) a sequence that creates a knockout of a gene; (vi) an expressible selection marker; (viii) inducible promoter; (ix) landing pad; or (x) any combination of i-ix.
47. The use of any of the preceding paragraphs, wherein the animal, cell, or embryo is heterozygous for the genetic modification made by the first HDR template.
48. The use of any of the preceding paragraphs, wherein the genetic modification is a modification of one or more tumor suppressor genes.
49. The use of any of the preceding paragraphs, applied to make a modified NF1 and/or TP53 site.
50. The use of any of the preceding paragraphs, further comprising adjusting a ratio of the first HDR template to the second HDR template.
51. The use of any of the preceding paragraphs, with the first template comprising a first site for a first restriction enzyme and the second template comprising a second site for a second restriction enzyme.
52. Use of a cell or embryo having one or more modified tumor suppressor genes to clone an animal therefrom.
53. Use of a cell or embryo of any of the preceding paragraphs, wherein the one or more tumor suppressor genes are heterozygously modified.
54. The use of the cell or embryo of any of the preceding paragraphs, wherein the modification comprises a modification of an NF1 gene and/or a TP53 gene.
55. The use of the cell or embryo of any of the preceding paragraphs wherein the one or more modifications are made in cis.

### REFERENCES

1. Rasmussen S A and Friedman J M. NF1 gene and neurofibromatosis 1. American journal of epidemiology. 2000; 151(1):33-40.
2. Upadhyaya M, Kluwe L, Spurlock G, Monem B, Majounie E, Mantripragada K, Ruggieri M, Chuzhanova N, Evans D G, Ferner R, Thomas N, Guha A and Mautner V. Germline and somatic NF1 gene mutation spectrum in NF1-associated malignant peripheral nerve sheath tumors (MPNSTs). Human mutation. 2008; 29(1):74-82.
3. Bikowska-Opalach B and Jackowska T. [Neurofibromatosis type 1—description of clinical features and molecular mechanism of the disease]. Medycyna wieku rozwojowego. 2013; 17(4):334-340.
4. Vogel K S, Klesse L J, Velasco-Miguel S, Meyers K, Rushing E J and Parada L F. Mouse tumor model for neurofibromatosis type 1. Science. 1999; 286(5447):2176-2179.
5. Cichowski K, Shih T S, Schmitt E, Santiago S, Reilly K, McLaughlin M E, Bronson R T and Jacks T. Mouse models of tumor development in neurofibromatosis type 1. Science. 1999; 286(5447):2172-2176.
6. Rasmussen S A, Overman J, Thomson S A, Colman S D, Abernathy C R, Trimpert R E, Moose R, Virdi G, Roux K, Bauer M, Rojiani A M, Maria B L, Muir D and Wallace M R. Chromosome 17 loss-of-heterozygosity studies in benign and malignant tumors in neurofibromatosis type 1. Genes, chromosomes & cancer. 2000; 28(4):425-431.
7. Carlson D F, Tan W, Lillico S G, Stverakova D, Proudfoot C, Christian M, Voytas D F, Long C R, Whitelaw C B and Fahrenkrug S C. Efficient TALEN-mediated gene knockout in livestock. Proceedings of the National Academy of Sciences of the United States of America. 2012; 109(43): 17382-17387.
8. Servin B, Faraut T, lannuccelli N, Zelenika D and Milan D. High-resolution autosomal radiation hybrid maps of the pig genome and their contribution to the genome sequence assembly. BMC genomics. 2012; 13:585.
9. Tan W, Carlson D F, Lancto C A, Garbe J R, Webster D A, Hackett P B and Fahrenkrug S C. Efficient nonmeiotic allele introgression in livestock using custom endonucleases. Proceedings of the National Academy of Sciences of the United States of America. 2013; 110(41):16526-16531.
10. Rasmussen S A and Friedman J M. NF1 gene and neurofibromatosis 1. American journal of epidemiology. 2000; 151(1):33-40.
11. Carlson D F, Tan W, Lillico S G, Stverakova D, Proudfoot C, Christian M, Voytas D F, Long C R, Whitelaw C B and Fahrenkrug S C. Efficient TALEN-mediated gene knockout in livestock. Proceedings of the National Academy of Sciences of the United States of America. 2012; 109(43): 17382-17387.
12. Cermak T, Doyle E L, Christian M, Wang L, Zhang Y, Schmidt C, Baller J A, Somia N V, Bogdanove A J and Voytas D F. Efficient design and assembly of custom TALEN and other TAL effector-based constructs for DNA targeting. Nucleic acids research. 2011; 39(12):e82.
13. Guschin D Y, Waite A J, Katibah G E, Miller J C, Holmes M C and Rebar E J. A rapid and general assay for monitoring endogenous gene modification. Methods in molecular biology. 2010; 649:247-256.

1. A swine or a cell or an embryo comprising a genomically modified NF1 gene and/or a modified TP53 gene.

2. The swine or cell or embryo of claim 1, wherein the modified NF1 gene comprises a modification at a location that is the equivalent of the arginine 1947 in human.

3. The swine or cell or embryo of claim 1, wherein the modified NF1 gene and/or the modified TP53 gene is modified to include a premature stop codon.

4. The swine or cell or embryo of claim 1, having a heterozygous modification of the NF1 gene.

5. The swine or cell or embryo of claim 4, having a heterozygous modification of the TP53 gene.

6. The swine or cell or embryo of claim 1, having a modification of both the NF1 gene and the TP53 gene.

7. The swine or cell or embryo of claim 6, wherein the modifications are in cis.

8. The swine or cell or embryo of claim 7, wherein one allele of the NF1 gene is a wildtype allele.

9. The swine or cell or embryo of claim 7, wherein one allele of the TP53 gene is a wildtype allele.

10. The swine or cell or embryo of claim 8, wherein one allele of the NF1 gene is a wildtype allele, except the wildtype NF1 allele has one or more silent mutations.

11. The swine or cell or embryo of claim 9, wherein one allele of the TP53 gene is a wildtype allele, except the wildtype TP53 allele has one or more silent mutations.

12. The swine or cell or embryo of claim 10, wherein the one or more silent mutations provides a site of attack for one or more restriction enzymes.

13. The swine or cell or embryo of claim 11, wherein the one or more silent mutations provides a site of attack for one or more restriction enzymes.

14. The swine or cell or embryo of claim 1, comprising one or more modifications that, provides a site of attack for a restriction enzyme.

15. The swine or cell or embryo of claim 1 being a miniature pig and/or ossabaw pig and/or landrace pig and/or founder and/or F1.

16. The cell of claim 1 being primary and/or swine and/or low passage.

17. The cell of claim 1 being a zygote, oocyte, gamete, sperm, or a member of an embryo/blastomere.

18. A method of making a cell swine or embryo of claim 1, comprising use of a targeted endonuclease and/or homology dependent repair template.

19. A method of making an animal, cell, or embryo comprising introducing into a cell or an embryo:
a targeted endonuclease directed to a target chromosomal DNA site;
a first HDR template homologous to the target chromosomal DNA site that comprises a first sequence that is exogenous to the target chromosomal DNA site; and
a second HDR template homologous to the target chromosomal DNA site that comprises a second sequence that is exogenous to the target chromosomal DNA site.

20. The method of claim 19, wherein the second sequence is identical to the target DNA chromosomal site.

21. The method of claim 19, wherein the second sequence has 99% identity to the target DNA chromosomal site.

22. The method of claim 20, wherein the second sequence has a 95% identity to the target DNA chromosomal site.

23. The method of claim 19, wherein the second sequence is identical to the target DNA chromosomal site except for (i) one or more silent mutations (ii) a number of bases ranging from 1-5.

24. The method of claim 19, wherein the second sequence is identical to the target DNA chromosomal site except for a change in sequence that allows for cleavage by one or more restriction enzymes.

25. The method of claim 19, wherein the exogenous sequence comprises, or is: (i) an allele found in nature; (ii) an allele found in the same species; (iii) an allele found in another breed of the same species; (iv) an allele from a different species; (v) a sequence that creates a knockout of a gene; (vi) an expressible selection marker; (viii) inducible promoter; (ix) landing pad; or (x) any combination of i-ix.

26. The method of claim 19, wherein the animal, cell, or animal is heterozygous for the genetic modification made by the first HDR template.

27. The method of claim 19, applied to make a modified NF1 and/or TP53 site.

28. The method of claim 19, applied to make a modified tumor suppressor gene.

29. The method of claim 19, further comprising adjusting a ratio of the first HDR template to the second HDR template.

30. The method of claim 19, with the first template comprising a first site for a first restriction enzyme and the second template comprising a second site for a second restriction enzyme.

31. The method of 19, comprising screening a plurality of cells for a genetic modification comprising identifying cells that comprise the first site and the second site.

32. An animal or a cell or an embryo comprising one or more genomically modified tumor suppressor genes, said gene being heterozygously modified.

33. The animal or cell or embryo of claim 32, made by the method of claim 19.

34. The animal or cell or embryo of claim 33, wherein one allele of the modified gene is a wildtype allele, except the wildtype NF1 allele has one or more silent mutations.

35. The animal or cell or embryo of claim 34, wherein the one or more silent mutations provides a site of attack for a restriction enzyme.

36. The animal or cell or embryo of claim 34, comprising a modification at one or both alleles of the one or more genes that provides a site of attack for one or more restriction enzymes.

37. The animal or cell or embryo of claim 34, being a livestock, cattle, swine, a miniature pig and/or ossabaw pig and/or Landrace pig and/or founder and/or F1.

38. The cell of claim 32, being primary and/or animal and/or low passage.

39. The cell of claim 32, being a zygote, oocyte, gamete, sperm, or a member of an embryo/blastomere.
